(19) United States
(12) Patent Application Publication
(10) Pub. No.: US 2005/0118564 A1
(43) Pub. Date: Jun. 2, 2005

(54) Methods and compositions for identification and therapeutic use of genes involved in vascular and proliferative diseases

(76) Inventors: Sivaram Pillarisetti, Norcross, GA (US); Uday Saxena, Atlanta, GA (US)

Correspondence Address: WOMBLE CARLYLE SANDRIDGE & RICE, PLLC, P.O. BOX 7037, ATLANTA, GA, 30357-0037, US

(21) Appl. No.: 10/500,769

(22) PCT Filed: Jan. 31, 2003
(86) PCT No.: PCT/US03/02940

Related U.S. Application Data
(60) Provisional application No. 60/353,415, filed on Jan. 31, 2002.

(51) Int. Cl. C12Q 1/00; G06F 19/00; G01N 33/48; G01N 33/50

(52) U.S. Cl. 435/4; 702/19

(57) ABSTRACT

The present invention comprises compositions and methods for identification of genes involved in vascular diseases using human endothelial and smooth cell “artery wall” models to capture the cell-cell and cell-matrix communications that are operative in normal and diseases conditions. The present invention further comprises the capture of the complexity of multiple chronic pathologies that coexist in humans by simultaneous treatment of the cells with disease specific stimuli and the identification and use of genes that are regulated by such multiple established disease mediators. The present invention additionally comprises compositions and methods of treatment of vascular disease.

### TECHNICAL FIELD

This application relates to compositions and methods for identification and therapeutic use of genes and gene products involved in vascular and proliferative diseases for the treatment of vascular and proliferative diseases and related disorders.

### BACKGROUND OF THE INVENTION

Many humans and animals have limited lifespans and lifestyles because of conditions relating to lifestyle choices, such as diet and exercise, or because of genetic predispositions for the development of particular diseases. For example, abnormal vascular smooth muscle cell proliferation in blood vessels is thought to contribute to the pathogenesis of vascular occlusive lesions, including arteriosclerosis, atherosclerosis, restenosis, and graft atherosclerosis after organ transplantation. Vascular smooth muscle cell proliferation is a common consequence of endothelial injury and is believed to be an early pathogenetic event in the formation of atherosclerotic plaques.

The blood vessel wall supplies blood and nutrients to various organs and tissues. The vessel wall is composed of an outer lining of endothelial cells, a sub-endothelial cell matrix and underlying smooth muscle cells. The endothelium is a monolayer of cells, which serves as a sieve between blood components and the vasculature and underlying cells and matrix. While not wishing to be bound to any particular theory, the endothelium is currently believed to play a role in maintenance of non-thrombogenic, non-adherent surfaces; regulation of endothelial cell transport of circulating biomacromolecules and nutrients; vasomotor tone through the production of bioactive molecules; and growth and remodeling in areas of neovascularisation and inflammatory response.

While inflammation is a normal immune response, chronic inflammation leads to complications and ongoing system damage. The complications and ongoing system damage are due to the interactions of unknown cellular factors. In particular, chronic inflammation can cause endothelial damage resulting in vascular complications. Because blood vessels are present in all tissue, blood vessel dysfunction can result in a number of diseases. Coronary artery, cerbrovascular and peripheral vascular disease resulting from atherosclerotic and thromboembolic macroangiopathy are the primary causes of mortality in chronic inflammatory diseases. Uncontrolled proliferation or apoptosis of smooth muscle cells may also lead to disease conditions (4) such as atherosclerosis, arthritis, asthma, restenosis, and diabetic nephropathy.

Control or modulation of factors produced by the body in response to injury, surgery, metabolic factors or feedback mechanism dysfunction has long been a treatment target. One disease that is rapidly growing in the industrialized countries is the occurrence of diabetes and all of its attendant sequellae. One of the factors important in the damage associated with diabetes is the presence of glycated proteins.

Atherosclerosis is a disease that includes both uncontrolled inflammation and proliferative components. It is the most prevalent of vascular diseases and is the principal cause of heart attacks, stroke and death in the United States. Atherosclerosis involves both endothelial cells and smooth muscle cells.

Percutaneous coronary artery intervention (PTCA) procedures are the most common in-patient hospital procedure in the United States. Patients undergo PTCA to reduce the amount of constriction in a coronary artery due to plaque formation. According to the American Heart Association, about one-third of the patients that undergo balloon angioplasty have restenosis of the widened segment of the vessel within approximately 6 months. It may be necessary to perform another angioplasty or coronary artery bypass surgery on restenosed arteries. A key feature of restenosis is an injury response that results in activation of an inflammatory cascade and remodeling of the cells both inside and outside the carotid artery wall. This includes excessive growth of connective tissue and smooth muscle into the lumen of the artery known as neointimal hyperplasia. Currently there are no effective pharmacological treatments available that control the pathogenesis of vascular occlusive lesions, such as, but not limited to, arteriosclerosis, atherosclerosis, restenosis, and graft atherosclerosis after organ transplantation. Identification of effective therapeutics with minimal side effects will restore quality of life without requiring additional surgical procedures such as coronary artery bypass surgery.

Another major area of unwanted cellular growth is cancer or oncological conditions. Many therapies have been used and are being used in an effort to restore health or at least stop the unwanted cell growth. Many times, individual therapeutic agents can have an effect, but often, therapeutic regimes require combinations of different pharmacological agents with treatments such as surgery or radiation.

Many studies aimed at defining the role of particular gene products presumed to be involved in the excessive inflammatory or proliferative response have been conducted in the past. These approaches used either endothelial cells or smooth muscle cells in isolation. Secondly, the cells were treated with only one stimulus e.g. a single cytokine. However, such approaches are too simplistic and do not take into account the complexity of interactions between the endothelial cell and smooth muscle cell and the matrix as described in the cell-matrix and cell-cell communication system. Furthermore these approaches do not consider the effect of two chronic diseases such as atherosclerosis and Type II diabetes, an increasingly common occurrence. Single and multiple disease progression involves the interaction of varied stimuli, activation/inactivation of multiple signaling pathways and the up-regulation or down regulation of a variety of genes. Therefore, the one cell type-one stimulus approach cannot identify the full panoply of gene products that are involved in the disease process, much less identify those which may serve as therapeutic targets for the diagnosis and treatment of various forms of vascular and proliferative disease.

Despite the importance of cell-cell interaction and cell-matrix interactions in determining the onset and progression of many disorders, no method is currently available for identification of endothelial cell or smooth muscle cell genes in a cell culture system that captures the complexity of cell-cell or cell-matrix interactions found in vivo. Further, no method is currently available for identification of genes in a cell culture system which are responsive to specific singular or multiple disease stimuli. There is therefore a need for the development of compositions and methods to identify therapeutically relevant genes in an in vitro system that accurately captures the complexity of disease processes found in vivo, in particular a system for the interactions found among and between endothelial cells and smooth muscle cells.

### SUMMARY OF THE INVENTION

The present invention is directed to methods and compositions that have utility in diagnosing, treating and preventing pathological conditions. The present invention also comprises methods and compositions for the treatment and diagnosis of vascular and proliferative disease, including but not limited to, atherosclerosis, arthritis, asthma, restenosis, acute inflammation, vascular inflammatory disease, chronic inflammation, angiopathy, myocarditis, nephritis, Crohn's disease and diabetic nephropathy. Specifically, the invention comprises the identification of genes and gene products which may be differentially expressed in vascular and proliferative disease states, relative to their expression in normal, or non disease states. The present invention further comprises screening methods to identify compounds and compositions which have diagnostic or therapeutic uses.

The present invention also comprises methods and compositions for prognostic and diagnostic evaluation of inflammatory and proliferative related disease conditions, and for the identification of individuals exhibiting a predisposition to such conditions. Furthermore, the invention provides methods for evaluating the efficacy of specific drugs, and monitoring the progress of patients being treated for vascular, proliferative and oncogenic related diseases.

The present invention additionally comprises methods and compositions for the identification of compounds that modulate the expression of genes or the activity of gene products involved in vascular and proliferative disease, as well as methods for the treatment of vascular and proliferative disease, which may involve the administration of the identified compounds to individuals exhibiting vascular and proliferative disease symptoms or tendencies.

The present invention further comprises methods and compositions for the identification of compounds that modulate the expression of genes or the activity of gene products involved in proliferative or inflammatory disease.

The present invention also comprises, in part, in vitro methods involving vascular and proliferative disease elements coupled with gene expression arrays. In contrast to approaches that merely evaluate the expression of a given gene product presumed to play a role in a disease process, the methods and compositions used herein permit the identification of all genes, whether known or novel, that are expressed or repressed in the disease condition, as well as evaluation of their regulation by multiple stimuli and function during disease progression. This comprehensive approach and evaluation permits the discovery of novel genes and gene products, as well as the identification of an array of genes and gene products (whether novel or known) involved in novel pathways that play a major role in the disease pathology. Thus, an embodiment of the present invention allows one to define targets useful for diagnosis, monitoring, rational drug screening and design, and/or other therapeutic intervention. Other aspects of the present invention comprise compositions and methods for microarray devices. Such microarray devices and methods comprise a variety of microarrays that may be used, for example, to study and monitor gene expression in response to treatment with the compounds of the present invention. The microarrays may comprise nucleic acid sequences, carbohydrates or proteins that are determinative for specific cells, tissues, species, disease states, prognoses, disease progression, or any other combination of molecules that can be used to determine an effect of one or more of the compounds of the present invention.

An embodiment of the present invention comprises methods and compositions comprising compounds of the present invention for the treatment and prevention of conditions or diseases that have as an aspect of the disease or condition, inflammation. Methods of treatment comprise administration of compositions comprising compounds having at least the activity of modulating inflammatory reactions that are components of biological conditions including, but not limited to, vascular complications of type I and type II diabetic-induced vasculopathies, other vasculopathies, microangiopathies, renal insufficiency, Alzheimer's syndrome, and inflammation-induced diseases such as atherosclerosis. An aspect of the present invention comprises methods and compositions for the treatment of diseases, preconditions or pathologies associated with inflammatory cytokines and other inflammation-related molecules.

The present invention also comprises pharmaceutical compositions comprising the compounds identified by the assays disclosed herein. Routes of administration and dosages of effective amounts of the compounds and pharmaceutical compositions are also disclosed. For example, the compounds of the present invention can be administered in combination with other pharmaceutical agents in a variety of protocols for effective treatment of disease.

The invention comprises methods for screening compounds and other substances for treating disease including, but not limited to atherosclerosis, arthritis, restenosis, chronic and acute inflammation, asthma, acute inflammatory diseases, vascular inflammatory disease, chronic inflammation, angiopathy, myocarditis, nephritis, Crohn's disease, type I and II diabetes and associated vascular pathologies, by assaying compound's ability to modulate the expression of the target genes identified by the assays disclosed herein or activity of the protein products of the target genes. The invention further comprises methods for screening compounds and other substances for treatment of proliferative disorders and inflammatory disease by assaying the ability of the compounds to modulate the expression of the target genes or activity of the protein products of the target genes. Such screening methods include, but are not limited to, assays for identifying compounds and other substances that interact with (e.g., bind to) the target gene/protein products.

In addition, the invention comprises methods for treating vascular and proliferative disease and related diseases by administering compounds and other substances that modulate the overall activity of the specific gene products. These compounds and other substances can affect such modulation either on the level of specific gene expression or specific protein activity.

### BRIEF DESCRIPTION OF THE FIGURES

FIG. 1 is a diagram illustrating an interaction of matrix, endothelial cells, and smooth muscle cells.

FIG. 2 is a chart showing the effect of insulin and/or TNFα on the production of MCP-1 in endothelial cells.

FIG. 3 is a chart showing the effect of insulin and/or TNFα on the production of VCAM-1 in endothelial cells.

FIG. 4 is a chart showing the effect of glycated human serum albumin and/or insulin on the production of MCP-1 in endothelial cells.

FIG. 5 is a chart showing the effect of insulin, TNFα or glycated human serum albumin on the production of MCP-1 and IL-6 in smooth muscle cells.

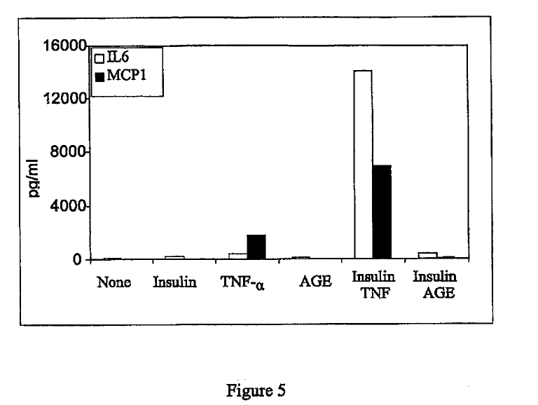

FIG. 6 is a chart showing the effect of TNFα on the production of IL-6 when endothelial cells and smooth muscle cells are grown as co-cultures.

FIG. 7 is a chart showing the effect of TNFα on the production of MCP-1 when endothelial cells and smooth muscle cells are grown in co-culture.

FIG. 8 is a chart showing the effect of IL-1β and glycated human serum albumin on the production of IL-6 when endothelial cells and smooth muscle cells are grown in co-culture.

### DETAILED DESCRIPTION OF THE INVENTION

The present invention relates to methods and compositions for the treatment, monitoring, prevention and diagnosis of vascular and proliferative disease, and related conditions, including, but not limited to, atherosclerosis, arthritis, restenosis, chronic and acute inflammation, asthma, acute inflammatory diseases, vascular inflammatory disease, chronic inflammation, angiopathy, myocarditis, nephritis, Crohn's disease, type I and II diabetes and associated vascular pathologies. The present invention further comprises the identification of genes and gene products whose expression is up or down regulated under conditions mimicking disease states, relative to their expression in normal, or non-disease states. Preferably the methods and compositions of the present invention comprise the identification and isolation of genes and gene products that interact with other gene products involved in vascular and proliferative diseases. The present invention also comprises the products of diagnostic, target, and pathway genes, as well as antibodies and nucleic acid antagonists to such gene products. The present invention additionally comprises screening methods to identify compositions for such therapeutic and diagnostic uses. The genes identified may be used diagnostically or as targets for therapeutic intervention. Additionally, methods are provided for the diagnostic monitoring of patients undergoing clinical evaluation for the treatment of inflammatory or proliferative related disorders, for monitoring the efficacy of compounds in clinical trials, and for identifying subjects who may be predisposed to vascular and proliferative disorders.

An embodiment of the present invention comprises the use of endothelial cells and smooth muscle cells separately in vitro to determine the effects of disease specific stimuli on gene expression surveyed by expression microarrays (Table 1, elements 1 and 2).

TABLE 1

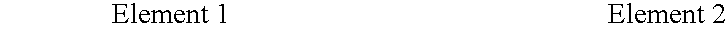

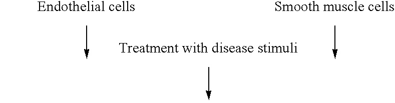

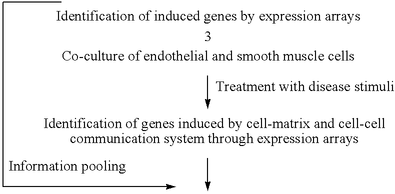

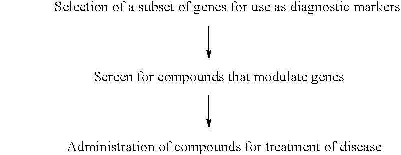

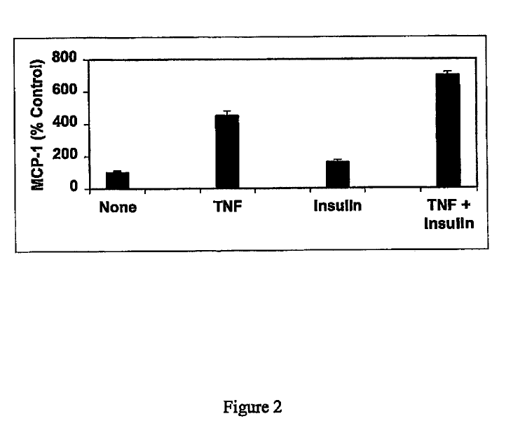

The present invention further comprises the use of endothelial cells and smooth muscle cells in contact co-cultures in which the endothelial cells and smooth muscle cells may be layered or otherwise cultured together and wherein the effects of multiple stimuli on gene expression are determined (FIG. 2, element 3); and, isolation of a subset of genes induced by cell-cell and cell-matrix systems that are common to all three elements and thus are most likely to be involved in disease processes. In all three elements the cells in culture will be exposed to a variety of disease specific stimuli simultaneously so as to recreate the complexity of the disease processes. Such an approach will not only uncover induced genes but also new signaling pathways leading to gene induction.

Though not wishing to be bound, it is currently believed that endothelial cells and smooth muscle cells in the blood vessel wall communicate through molecules in the sub-endothelial matrix. The sub-endothelial matrix is a complex organization of protein and carbohydrate molecules that are secreted by both the endothelial and smooth muscle cells. The molecules are the basis of signaling between these two cell types and the rest of the body. Certain disease states, including, but not limited to atherosclerosis, arthritis, restenosis, chronic and acute inflammation, asthma, acute inflammatory diseases, vascular inflammatory disease, chronic inflammation, angiopathy, myocarditis, nephritis, Crohn's disease, type I and II diabetes and associated vascular pathologies, can cause alterations in the types or amounts of these signaling molecules. Alterations in the signaling molecules received or transmitted by endothelial cells can result in inflammatory disorders, such as diabetic vasculopathy or arthritis. Alterations in the signaling molecules received or transmitted by the smooth muscle cells can lead to proliferative disorders such as restenosis or cancer.

The cell matrix signaling pathway (CMS pathway) as used herein means the sophisticated communication and feedback between endothelial cells and smooth muscle cells. The endothelial cells form the outer lining of the blood vessel and are believed to perform a gatekeeper function. These cells determine the types of molecules that are transported from blood to the tissues. Underlying the endothelial cells are the smooth muscle cells which provide mechanical strength to the blood vessel and are essential to the overall integrity of the blood vessel. Both the endothelial cells and the smooth muscle cells, manufacture and secrete molecules into the space surrounding them to form a matrix or scaffold that holds the blood vessel together.

The information system for endothelial cells and smooth muscle cells is based upon molecules secreted into the matrix. These messengers dictate the growth of these cells, the movement of these cells, and the overall function (or dysfunction) of these cells. This pathway controls the function of the blood vessels. Through control of blood vessels, this pathway controls the function of the surrounding tissues. The location of the affected blood vessel within the body determines which of a variety of tissue functions can be effected.

The present invention comprises methods for identifying CMS pathway induced genes that are modulated during vascular and proliferative diseases and related disorders comprising separately exposing smooth muscle cells and endothelial cells to vascular and proliferative disease stimuli including, but not limited to, AGE, insulin, IL-1β and TNF-α. The vascular and proliferative disease stimuli can be added alone or in combination. The smooth muscle cells and endothelial cells are cultured together or may be stacked or otherwise assembled into more complex structures to mimic blood vessels so that the smooth muscle cells are layered over the endothelial cells or vice versa in co-culture. The smooth muscle cell and endothelial muscle cell co-cultures are also exposed to vascular and proliferative disease stimuli and the expression level of vascular and proliferative disease markers expressed by the cells is measured and compared both to each other and to the expression level of the vascular and proliferative disease markers in cells which have not been exposed to vascular and proliferative disease stimuli. The vascular and proliferative disease markers include, but are not limited to, interleukin 6 (IL-6), interferon-inducible protein-10 (IP-10), monokine induced by gamma-interferon (MIG), interferon-inducible T-cell alpha chemoattractant (I-TAC). vascular adhesion molecule-1 (VCAM-1), monocyte chemoattractant protein-1 (MCP-1), fragments, genes, nucleotide sequences, and or control elements.

Test compounds such as oligonucleotides, ribozymes, antisense oligonucleotides, peptides, peptoids, small organic molecules or small inorganic molecules may also be added to the cell cultures to determine if the test compounds are effective in increasing the rate of expression of the disease markers or genes involved in the CMS pathway, decreasing the rate of expression, or if the compounds have no effect at all. The test compounds can increase the activity of the gene or protein product, decrease the activity of the gene or protein product or have no effect at all. The test compounds may have a variety of effects, including, but not limited to, binding to the CMS induced gene, blocking translation or binding to the protein product of the CMS induced gene, preventing it from acting or being acted upon.

The present invention additionally comprises methods of identifying other gene products which may be differentially expressed in vascular and proliferative disease states and related disorders comprising isolating cellular products from samples of patients with vascular or proliferative diseases or related disorders and comparing the levels of the cellular products to the levels in a sample from a individual who does not have a vascular or proliferative disease or related disorder. Cellular products that have elevated or depressed levels in comparison to controls may indicate additional disease markers.

The methods and compositions of the present invention may also be used to diagnose vascular and proliferative diseases or related disorders and comprise measuring or detecting the level of activity such as replication, transcription or translation of the CMS pathway induced genes or disease markers in a patient suspected of having a vascular or proliferative disease or related disorder and comparing the level of transcription to a control sample from an individual who does not have a vascular or proliferative disease or related disorder.

The present invention may additionally be used to diagnose a vascular or proliferative disease or related disorder comprising detecting the level of the protein product from a CMS pathway induced gene or the protein activity in a patient or patient sample and comparing it to the level of protein product or protein activity in a control sample. The above methods may also be used to monitor the efficacy of a treatment program or the response of an individual in a clinical trial.

A further embodiment of the present invention comprises compositions and methods for microarray devices. Such microarray devices and methods comprise a variety of microarrays that may be used, for example, to study and monitor gene expression in response to treatment with the compounds of the present invention. As used herein, the term “microarray,” refers to an ordered spatial arrangement of immobilized biomolecular probes arrayed on a solid supporting substrate. Preferably, the biomolecular probes are immobilized on second linker moieties in contact with polymeric beads, wherein the polymeric beads are immobilized on first linker moieties in contact with the solid supporting substrate. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors). The microarrays may comprise nucleic acid sequences, carbohydrates or proteins that are determinative for specific cells, tissues, species, disease states, prognoses, disease progression, or any other combination of molecules that can be used to determine an effect of one or more of the compounds of the present invention. Other embodiments of the present invention comprise methods using databases and computer applications to analyze the expression rate of the CMS pathway induced genes.

The present invention also comprises methods and compositions for diagnosing a vascular or proliferative disease or a related disorder comprising measuring the protein product level or the protein activity present in a sample and comparing it to a control such that if the level of protein or protein activity detected is different from the control, the difference is indicative of a vascular, proliferative or an oncogenic disease or related disorder. The protein product can be measured by assay systems known to those skilled in the art, including but not limited to, competitive and non-competitive assay systems using techniques such as microarrays, radioimmunoassays, ELISA (enzyme linked immunosorbent assay), “sandwich” immunoassays, precipitin reactions, gel diffusion precipitin reactions, immunodiffusion assays, agglutination assays, complement fixation assays, immunoradiometric assays, fluorescent immunoassays, protein A immunoassays, and immunoelectrophoresis assays, to name but a few.

CMS induced genes include “diagnostic genes,” and/or “target genes.” “Diagnostic genes,” as used herein, refers to a gene whose expression pattern may be utilized as part of a prognostic or diagnostic disease evaluation, or which, alternatively, may be used in methods for identifying compounds useful for the treatment of vascular or proliferative diseases. CMS induced genes include, but are not limited to, those that produce products such as interleukin 6 (IL-6), interferon-inducible protein-10 (IP-10), monokine induced by gamma-interferon (MIG), interferon-inducible T-cell alpha chemoattractant (I-TAC). vascular adhesion molecule-1 (VCAM-1), or monocyte chemoattractant protein-1 (MCP-1).

“Target gene”, as used herein, refers to a differentially expressed gene involved in disease such that modulation of the level of target gene expression or of target gene product activity may act to ameliorate a disease condition. Compositions of the present invention comprise compounds that modulate target gene expression or activity of the target gene product which can be used in methods of treatment of disease.

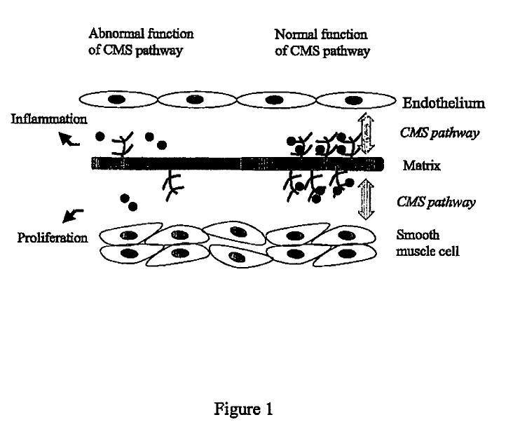

Both endothelial cells and smooth muscle cells actively participate in maintaining homeostasis by regulating the expression of key genes in response to the environment. The endothelial cells and smooth muscle cells are in constant communication with each other via cell surface receptors, secreted growth factors, cytokines, hormones and bioactive lipids (5-10). Cell behaviors that feature prominently in normal function and disease are shape, adhesion, migration, differentiation, proliferation and cell death. Cell behaviors and communications or intra and inter-cell factors are mediated in part by interactions of cells with an extracellular environment, consisting in most cases of a heterogeneous, macromolecular matrix that is specific, not only to a given tissue, but to cells contributing to the compartmentalization of organs and tissues. These cells reside in a protein network, the extracellular matrix, which they secrete and mold into the intercellular space (11). Though not wishing to be bound to any particular theory, it is believed that the effects of the matrix are mediated in part by integrins, a family of cell surface receptors that attach cells to the matrix and mediate mechanical and chemical signals from it (12-15). These signals regulate the activities of cytoplasmic kinases, growth factor receptors, and ion channels and control the organization of the intracellular actin cytoskeleton. Many integrin signals converge on cell cycle regulation, directing cells to live or die, to proliferate, or to exit the cell cycle and differentiate. Both cell-matrix and cell-cell communication systems consist in part of receptor-ligand interactions resulting in signal transduction for the initiation/modification of gene expression. (See FIG. 1). Any disruption or modulation of the cell-matrix and cell-cell communication system triggers abnormal gene expression leading to a disease state (15-17).

An example of cell-matrix and cell-cell communication system related dysfunction leading to disease is the production of the growth modulating peptide endostatin (18). Endostatin is a carboxyl-terminal proteolytic cleavage product of collagen, a key matrix component, which directly effects endothelial cell function and disease. This peptide has been shown to significantly inhibit endothelial cell proliferation and migration and new vessel development (anti-angiogenesis) important for tumor growth. Regulation of the proteolysis of the collagen to release active angiostatin is not yet fully understood, although this function has been ascribed to proteases.

While not wishing to be bound to any particular theory, acute and chronic inflammation are hallmark vascular diseases that may be triggered by abnormal functioning of the cell-cell or cell-matrix system. These disease processes begin with adhesion of leukocytes to the endothelium. Leukocyte recruitment to the endothelium is started when inducible adhesion molecule receptors on the surface of endothelial cells interact with counter receptors on immune cells. Vascular endothelial cells determine which type of leukocytes (monocytes, lymphocytes, or neutrophils) are recruited, by selectively expressing specific adhesion molecules, such as vascular cell adhesion molecule-1 (VCAM-1), intracellular adhesion molecule-1 (ICAM-1), and E-selectin. For example, because of the selective expression of VLA-4 on monocytes and lymphocytes, but not neutrophils, VCAM-1 is important in mediating the selective adhesion of mononuclear leukocytes.

Glycated proteins and advanced glycation end products (AGE) contribute to cellular damage, by at least two major mechanisms; modulation of cellular functions through interactions with specific cell surface receptors, and alteration of the extracellular matrix leading to the formation of protein cross-links. AGE increases lipoprotein oxidisability and atherogenicity. Studies suggest that glycated protein and AGE interactions with cells may promote inflammatory processes and oxidative cellular injury. Its binding to matrix proteins induces synthesis of cytokines and activates cellular messengers. Diseases where glycated protein and AGE accumulation is a suspected etiological factor include vascular complications of diabetes, microangiopathies, renal insufficiency, atherosclerosis and Alzheimer's disease.

Atherosclerosis is a disease that includes both uncontrolled inflammation and proliferative components. Several risk factors such as hyperlipidemia, hypertension and players such as growth factors and cytokines are involved in the initiation progression and maturation of an atherosclerotic lesion. In the earliest stages of the atherosclerotic lesion formation, there is a localized endothelial expression of vascular cell adhesion molecule-1 (VCAM-1) and selective recruitment of mononuclear leukocytes that express the integrin counter receptor very late antigen 4 (VLA-4). Protein chemoattractants like monocyte chemoattractant protein-1 (MCP-1) secreted by endothelial cells and smooth muscle cells then guide the monocytes into the sub-endothelial location. Subsequent conversion of monocytes to foamy macrophages results in the synthesis of a wide variety of inflammatory cytokines, growth factors, and chemoattractants that help propagate the leukocyte and platelet recruitment, smooth muscle cell proliferation, endothelial cell activation, and extracellular matrix synthesis characteristic of maturing atherosclerotic plaques. From initiation of the atherosclerotic lesion to maturation into a plaque, there exists a series of cell-cell and cell-matrix communications that result in abnormal expression of genes.

This complex process becomes even more intricate when two separate diseases overlap, e.g. a diabetic patient having atherosclerosis. In this situation several risk factors work at the same time to synergistically affect the disease process. Indeed, diabetics have an increased predisposition to micro- and macrovascular diseases such as nephropathy and atherosclerosis. Chronic diabetes is associated with a variety of stimulatory diabetic factors that can result in endothelial dysfunction. Hyperglycemia, hyperlipidemia and hyperinsulinemia found in diabetics might all act on the vascular cells to induce expression of genes important in atherosclerotic lesion formation. Hyperglycemia and exposure to high glucose levels may directly modulate endothelial and smooth muscle cell properties. In addition, excess plasma or tissue glucose levels can result in formation of non-enzymatic glucose-adducts with lysine residues in proteins called glycated proteins and advanced glycation end products. These stable forms of glycated proteins have cytokine-like inflammatory properties. Hyperlipidemia is often associated with diabetes in the form of high triglyceride and high levels of low-density lipoproteins (LDL). It is well established that oxidatively modified forms of LDL (ox-LDL) are biologically active and can affect endothelial/smooth muscle cell gene expression profiles. Lastly, in some patients with Type II diabetes, high circulating levels of insulin (hyperinsulinemia) are found and hyperinsulinemia is rapidly emerging to be an important risk factor. All three genes are up-regulated in response to disease stimuli and are specifically expressed in vascular cells.

An aspect of the present invention comprises methods and compositions for the treatment of diseases, preconditions or pathologies associated with inflammatory cytokines including, but not limited to TNFα, IL-6, VCAM-1, AGE-induced MCP-1, selecting, IP-10, MIG and I-TAC. These cytokines are thought to effect the pathogenesis of atherosclerosis and the development of diabetic vasculopathy in type II diabetes.

The present invention further comprises methods for identifying compounds useful in the treatment of diseases affected by CMS pathway induced genes comprising adding vascular disease stimuli and a test compound with unknown effects on endothelial cells to a first cell culture of endothelial cells; adding vascular disease stimuli and a test compound with unknown effects on smooth muscle cells to a second cell culture of smooth muscle cells; adding vascular disease stimuli and a test compound to a third cell culture of endothelial cells and smooth muscle cells in co-culture; measuring the amount of vascular disease markers in the first, second and third test cultures and comparing the amount of the vascular disease makers in the first, second and third cell cultures to each other. The amount of vascular disease markers in the first, second and third cell cultures may also be compared to smooth muscle cells, endothelial cells and smooth muscle cells and endothelial cells in co-culture to which test compounds have been added, vascular stimuli have been added, or the no stimuli or test compounds have been added. The compounds may be added before, with, or after the stimuli.

The present invention further comprises methods of diagnosing vascular and proliferative diseases comprising measuring the level of transcription of CMS pathway induced genes present in a patient or patient sample and in a corresponding control sample and comparing the level of CMS pathway induced genes transcript in both samples, wherein if the level of transcript detected differs in the patient sample relative to the corresponding control sample, a vascular disease or a related disorder is diagnosed.

The present invention further comprises methods of diagnosing vascular and proliferative diseases and related disorders comprising measuring CMS pathway induced gene product protein level or protein activity present in a patient or patient sample and in a corresponding control sample and comparing the CMS pathway induced gene product protein level or protein activity in both samples, wherein if the level of protein or protein activity detected differs in the patient or patient sample relative to the corresponding control sample, a proliferative disease or an oncogenic related disorder is diagnosed.

The present invention additionally comprises methods for monitoring vascular and proliferative diseases and related disorders comprising measuring the amount of vascular disease markers in a patient sample. The patient is then treated for the vascular and proliferative disease or related disorder either through conventional means, or through the use of one of the compounds identified by the assays described herein and samples are taken from the patient periodically during the treatment and the amount of vascular disease markers are measured.

In the examples described herein, three human genes, specifically gene products are identified that are demonstrated to be genes induced by the cell-matrix and cell-cell communication system. Specifically IL6, MCP-1 and VCAM-1 genes are differentially regulated in co-cultures subjected to the cell-matrix and cell-cell communication system. Accordingly, methods are provided for the diagnosis, monitoring, screening for therapeutically effective compounds, and compositions and methods of treatment of vascular and proliferative diseases based upon the discoveries herein regarding the expression patterns of these genes.

The present invention comprises methods for identifying cell matrix signaling (CMS) pathway induced genes that are modulated during vascular disease and related disorder comprising adding vascular disease stimuli to a first cell culture of endothelial cells; adding vascular disease stimuli to a second cell culture of smooth muscle cells; adding vascular disease stimuli to a third cell culture of endothelial cells and smooth muscle cells in co-culture; growing a fourth cell culture of endothelial cells (control); growing a fifth cell culture of smooth muscle cells (control); measuring the amount of vascular disease markers in first, second, fourth and fifth cell cultures; and comparing the amount of the vascular disease markers in the cell cultures to each other. The smooth muscle cells may be layered over the endothelial cells in co-culture to simulate blood vessels. Furthermore, the smooth muscle cells may be layered over the endothelial cells in different ratios, in a range of 1:10 to 10:1 and including, but not limited to, 1:1, 1:2, 1:3, 1:4, 1:5, 1:6, 1:7, 2:1, 2:3, 2:5, or 2:7.

The characteristic differential regulation of genes such as IL-6, MCP-1 and VCAM-1 underlie methods for vascular and proliferative disease treatment strategies. For those up-regulated genes that have a causative effect on the disease conditions (target genes), treatment methods are contemplated designed to reduce or eliminate their expression. Alternatively, treatment methods also include inhibiting the activity of the protein products of these genes. For those up-regulated genes that have a protective effect, treatment methods also comprise enhancing the activity of the products of such genes. These treatment methods comprise the administration of compounds which have been identified by the present invention as affecting genes in the CMS pathway.

IL-6 is a pro-inflammatory cytokine that is believed to play a key role in the pathogenesis of diabetes and atherosclerosis (23). IL-6 also promotes the growth of renal mesangial cells thus contributing to nephropathy (24). The serum IL-6 level in diabetic subjects was significantly higher than in normal healthy controls (3.48+/−3.29 pg/ml vs 0.784+/−0.90 pg/ml, mean+/−SD) making the measurement of urinary and serum levels of IL-6 a useful indicator in the evaluation of atherosclerosis and nephropathy.

MCP-1, another pro-inflammatory cytokine, is found highly expressed in human atherosclerotic lesions and postulated to play a central role in monocyte recruitment into the arterial wall and developing lesions. Recent results show that MCP-1 is also a key pathogenic molecule in diabetic nephropathy (25). The levels of urinary MCP-1 in patients with the advanced stage were significantly higher than those in patients with the mild stage of the disease, or in healthy controls. The measurement of urinary MCP-1 is therefore useful in evaluating the degree of renal injuries and/or prognosis in patients with nephropathy.

Detecting expression of these genes in excess of normal expression provides for the diagnosis of vascular and proliferative diseases. Furthermore, in testing the efficacy of compounds, a decrease in the level of the expression of these genes corresponds to a return from a disease condition to a normal state, and thereby indicates a positive effect of the compound. The vascular and proliferative diseases that may be so diagnosed, monitored, and treated include but are not limited to atherosclerosis, arthritis, restenosis, chronic and acute inflammation, asthma, acute inflammatory diseases, vascular inflammatory disease, chronic inflammation, angiopathy, myocarditis, nephritis, Crohn's disease, type I and II diabetes and associated vascular pathologies.

The present invention further relates to the administration of at least one compound in a pharmaceutical composition by parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intacervical, intragastric, intrahepatic, intramyocardial, intraosteal, intapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal means.

The compositions described herein are also contemplated to include the pharmaceutical compositions containing compounds determined by the assays of the present invention that can further comprise at least one of any suitable auxiliary such as, but not limited to, diluent, binder, stabilizer, buffers, salts, lipophilic solvents, preservative, adjuvant or the like. Pharmaceutically acceptable auxiliaries are preferred. Examples and methods of preparing such sterile solutions are well known in the art and can be found in well known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the compound. Pharmaceutical excipients and additives useful in the present invention include, but are not limited to, proteins, peptides, amino acids, lipids, and carbohydrates. The pharmaceutical compositions comprising the compounds of the present invention can also include a buffer or a pH adjusting agent. Additionally, pharmaceutical compositions of the invention can include polymeric excipients/additives.

For oral administration, pharmaceutical compositions can be in the form of a tablet or capsule, such as discrete units such as capsules, cachets or tablets each containing a predetermined amount of the immunity linker molecules; as a powder or granules; as a solution or a suspension in an aqueous liquid or a non-aqueous liquid; or as an oil-in-water liquid emulsion or a water-in-oil emulsion and as a bolus, etc. A tablet may be made by compression or molding, optionally with one or more accessory ingredients. The tablets may be optionally coated or scored and may be formulated so as to provide a slow or controlled release of the active ingredient therein.

In addition, the compositions may be incorporated into biodegradable polymers allowing for sustained release of the immunity linker molecules, for example, the polymers being implanted for slow release of the immunity linker molecules. Biodegradable polymers and their uses are described, for example, in detail in Brem et al., 74 J. NEUROSURG. 441-46 (1991).

Formulations suitable for parenteral administration include aqueous and non-aqueous sterile injection solutions which may contain anti-oxidants, buffers, bacteriostats and solutes that render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the immunity linker molecules to be administered in a suitable liquid carrier. The liquid forms may include suitably flavored suspending or dispersing agents such as the synthetic and natural gums, for example, tragacanth, acacia, methylcellulose and the like. Formulations for rectal administration may be presented as a suppository with a suitable base comprising, for example, cocoa butter or a salicylate. Formulations suitable for vaginal administration may be presented as pessaries, tamports, creams, gels, pastes, foams or spray formulations containing in addition to the active ingredient such carriers as are known in the art to be appropriate.

The compounds may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nanoparticles and nanocapsules) or in macroemulsions. REMINGTON'S PHARMACEUTICAL SCIENCES (A. Osol ed., 16th ed, (1980)).

The present invention provides stable formulations as well as preserved solutions and formulations containing a preservative as well as multi-use preserved formulations suitable for pharmaceutical or veterinary use, comprising the immunity linker molecule compositions disclosed herein in a pharmaceutically acceptable formulation.

In general, the compositions identified by the assays disclosed herein may be used alone or in concert with therapeutic agents at appropriate dosages defined by routine testing in order to obtain optimal efficacy while minimizing any potential toxicity. The dosage regimen utilizing a compound of the present invention may be selected in accordance with a variety of factors including type, species, age, weight, sex, medical condition of the patient; the severity of the condition to be treated; the route of administration; the renal and hepatic function of the patient; and the particular composition or therapeutic agent employed. A physician or veterinarian of ordinary skill can readily determine and prescribe the effective amount of the immunity linker molecule required to prevent, counter, or arrest the progress of the condition.

The dosages of a composition determined by the assays disclosed herein may be adjusted when combined to achieve desired effects. Data obtained from cell culture assays and animal studies may be used in formulating a range of dosages for use in humans.

Methods are known in the art for determining effective doses for therapeutic and prophylactic purposes for the disclosed pharmaceutical compositions. More specifically, the pharmaceutical compositions may be administered in a single dose, or a single daily dose or the total daily dosage may be administered in divided doses of two, three, or four times daily. The dosage of the compositions may be varied over a wide range from about 0.0001 to about 1,000 mg per patient or until an effective response is achieved. The range may more particularly be from about 0.001 mg/kg to 10 mg/kg of body weight, about 0.1-100 mg, about 1.0-50 mg or about 1.0-20 mg, for adults (at about 60 kg). The compositions may be administered on a regimen of about 1 to about 10 times per day, for one or multiple days, or once a week or once a month, or until an effective response is achieved. The pharmaceutical compositions of the present invention may be administered at least once a week over the course of several weeks or months.

In addition, co-administration or sequential administration of the compositions of the present invention and other therapeutic agents may be desirable. A composition described herein can be administered before or after administration of a second therapeutic agent. The administration of a composition may occur anytime before or after the administration of the second therapeutic agent.

The terms “treatment,” “treating,” “treat” and the like are used herein to refer generally to obtaining a desired pharmacological and/or physiological effect. The effect may be prophylactic in terms of completely or partially transferring immunity from one antigen to another and/or may be therapeutic in terms of a partial or complete stabilization or cure for a disease and/or adverse effect attributable to the disease. “Treatment” as used herein covers using the immune response directed to one antigen for the control of another antigen or its effects such as any treatment of a disease in a subject, particularly a human, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The expression “therapeutically effective amount” refers to an amount of, for example, a composition determined by the assays disclosed herein, that is effective for preventing, ameliorating, treating or delaying the onset of a disease or condition.

A “prophylactically effective amount” refers to an amount of, for example, a composition determined by an assay disclosed herein that is effective for preventing a disease or condition.

As used herein and in the appended claims, the singular forms “a,” “an,” and “the” include plural reference unless the context clearly indicates otherwise. Thus, for example, reference to a “compound” is a reference to one or more such compounds and includes equivalents thereof known to those skilled in the art, and so forth. Unless defined otherwise, all technical and scientific terms used herein have the same meaning as commonly understood to one of ordinary skill in the art to which this invention belongs.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The present invention may be understood more readily by reference to the following examples. It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

### EXAMPLES

### Example 1

### Endothelial Cells Exposed to Multiple Disease-Specific Stimuli Respond With Markedly Abnormal Gene Expression

Human aortic endothelial cells (HAEC) were grown with culture medium (Basal medium from Clonetics) containing 0.2% albumin with or without 1 ng/ml TNF-α or 100 μg/ml glycated-human serum albumin (HAS) or 1 nM insulin for 16 hrs. Media were collected and the amounts of L-6 (DuoSet ELISA development kit—R&D Systems) and MCP-1 (Quantiline Human MCP-1 kit—R&D Systems) were measured. VCAM-1 expression was measured by incubating serum with a polyclonal goat anti-human VCAM-1 antibody (R&D Systems, 1:1000 dilution) followed by development with a rabbit anti-goat IgG-conjugated with horseradish peroxidase (HRP).

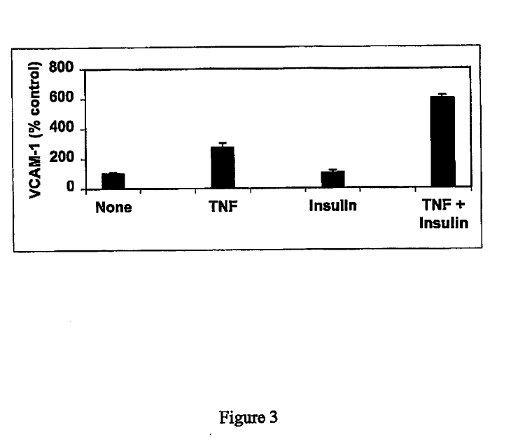

TNF-α alone increased MCP-1 secretion by about 400%. Insulin alone increased the secretion of MCP-1 by 40% (FIG. 3). More interestingly, insulin augmented the effect of TNF-α (MCP-1 induction=700%), indicating a synergistic effect of insulin on TNF-α-induced secretion of MCP-1.

Insulin addition alone had no effect on VCAM-1 expression, but similar to MCP-1 expression, addition of insulin together with TNFα markedly enhanced TNFα's effect on VCAM-1 gene expression from 2.8 fold to 6 fold. Thus MCP-1 and VCAM-1 expression were exacerbated by insulin.

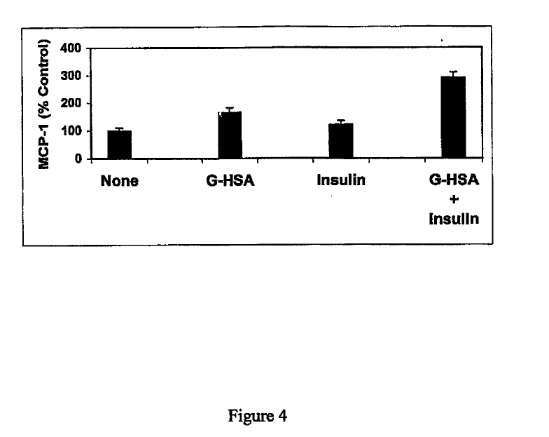

To examine whether atherogenic gene expression augmented by insulin is also found with another major player in diabetes, i.e. glucose-modified proteins, insulin was added together with glycated albumin (FIG. 4) to HAEC cells. G-HSA addition alone increased MCP-1 secretion by about 60%. Insulin augmented the effect of G-HSA (MCP-1 induction=300%), thus insulin augments MCP-1 expression activated by glycated albumin, another important regulator of diabetes-induced atherosclerosis.

### Example 2

### Smooth Muscle Cells Exposed to Multiple Disease-Specific Stimuli Respond With Markedly Abnormal Gene Expression

Smooth muscle cells were grown in culture medium (Basal medium from clonetics) containing 0.2% albumin. Aliquots of cells were treated with 1ng/ml TNF-α, insulin, or 1ng/ml TNFα and 1 nM insulin. Media were collected and the amount of IL-6 and MCP-1 secreted was determined as described in Example 1.

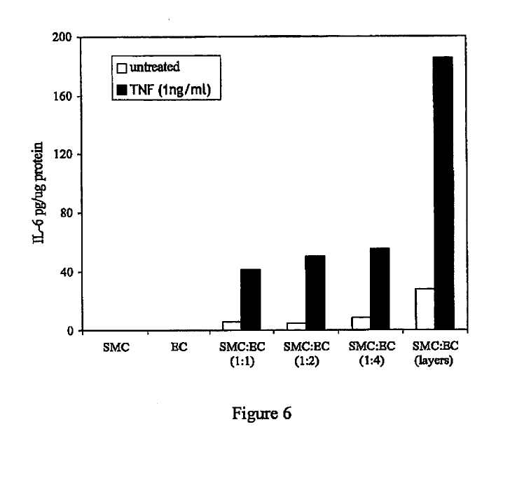

The synergistic effect of insulin and TNF-α was also seen with smooth muscle cells (FIG. 6). Insulin or TNF-α showed only moderate induction of IL-6 and MCP-1 in smooth muscle cells. When added together, there was a 4-(MCP-1) to 14-fold (IL-6) induction in the expression of IL-6 and MCP-1.

### Example 3

### Exacerbation of Inflammatory Gene Expression in Co-Cultures of Endothelial and Smooth Muscle Cells

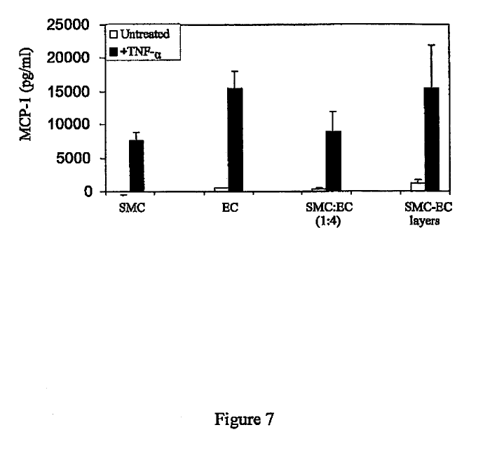

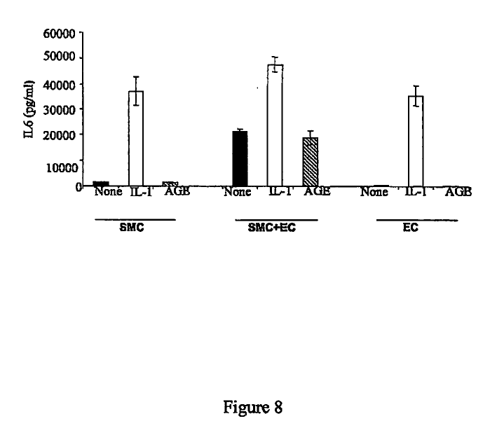

Endothelial and smooth muscle cells were grown together in culture media (Basal medium from Clonetics) containing 0.2% albumin. The cells had either nothing added, 1ng/ml TNFα, 1 nM insulin or TNFα and insulin. The media were collected and the amount of IL-6 and MCP-1 was measured. When these cells are co-cultured together, both basal and TNFα induced IL-6 expression was greatly enhanced (FIG. 6). This induction was seen both under conditions where endothelial and smooth muscle cells were mixed at different ratios (1:1, 1:2 and 1:4) or when smooth muscle cells were layered over endothelial cells to simulate blood vessel conditions in culture. The induction was more pronounced in the blood vessel-like culture model. This induction was not seen with MCP-1 (FIG. 7). Unlike TNFα, neither interleukin-1 nor glycated albumin/advanced glycation end products (AGE) showed enhanced induction in co-cultures of stacked smooth muscle cells and endothelial cells (FIG. 8).

Those skilled in the art will now see that certain modifications may be made to the invention here disclosed with respect to the illustrated embodiments, without departing from the spirit of the instant invention. And while the invention has been described above with respect to the preferred embodiments, it will be understood that the invention is adapted to numerous rearrangements, modifications, and alterations, all such arrangements, modifications, and alterations are intended to be within the scope of the appended claims.

### Example 4

### MICROARRAY ASSAY

### cDNA Probe Preparation

cDNA is prepared from cellular RNA s follows, 2.5 μg of cellular RNA is mixed with 2.0 μL Oligo dT (1 μg/μL 10-20 mer mixture) in a total volume of 10.0 μL and then placed at 70° C. for 10 minutes followed by brief chilling on ice for 2 minutes. To this mixture is added 6.0 μL 5× First Strand Buffer, 1.0 μL DTT (0.1 M), 1.5 μL dNTP mixture containing dATP, dGTP, and dTTP a;plo;′/ta concentration of 20 mM, 1.5 μL Reverse Transcriptase (Superscript II, RNase H.sup.-, Life Technologies, Rockville, Md.) and 10 μL .alpha(.sup.33 P)-dCTP (10 mCi/mL with a specific activity of 3000 Ci/mmol), in a total volume of 30 μL. This reaction mixture is incubated for 90 minutes at 37° C. The reaction mixture was diluted to a final volume of 100 μL with water, and the entire volume loaded on a BioSpin 6 chromatography column (BioRad, Hercules, Calif.). cDNA is purified from unincorporated nucleotides following the manufacturer's instructions. The efficiency of cDNA synthesis is calculated based on the radioactivity of the probe, and was generally between 10-15%. The entire probe mixture is used for hybridization.

Hybridization

Microarrays Were Obtained From Affymetrix (Calif.).

Prior to using new membranes, the membranes are washed for at least 5 minutes with gentle agitation in a boiling (95-100° C.) solution of 0.5% SDS. The arrays are prehybridized prior to use in hybridization roller bottles containing 5 mL MicroHyb hybridization solution. Blocking reagents, including 5.0 μg human Cot-1 DNA and 5.0 μg poly dA are added to the prehybridization mixture (wherein the Cot-1 DNA is first denatured by boiling for 3 minutes). Prehybridization is performed by incubating the membrane in prehybridization buffer at 42° C. for 4 to 6 hours.

Hybridization of radiolabeled cDNA prepared as described above to the prehybridized microarrays is performed as follows. The radiolabeled cDNA probe is denatured by boiling for 3 minutes and then rapidly chilled on ice for 2 minutes. The probe is introduced into the hybridization roller bottle containing the membrane and the prehybridization mixture. The membrane is hybridized to the probe by incubation overnight (12-18 hours) at 42° C. in a hybridization roller oven at 8-10 rpm. After the hybridization reaction is complete, the membranes are washed twice with 30 mL of 2×SSC containing 1% SDS at 50° C. for 20 minutes in hybridization oven at 12-15 rpm. The membranes are then washed in 1 mL of a solution of 0.5×SSC containing 1% SDS at room temperature for 15 minutes with gentle shaking. The washed membrane is then placed on a moist filter paper and wrap it with plastic wrap.

Hybridization is detected using a using a Cyclone phosphor imaging analyzer (Packard, Meriden, Conn.) by placing the hybridized membrane adjacent to the phosphor imaging screen. The membrane was exposed to the screen for a time sufficient to detect hybridization, following the manufacturer's instructions. Results are interpreted using Pathways™ software for analysis.

After exposure and data collection, the membranes are recovered for additional rounds of hybridization by stripping the hybridized radioactive probe. This is accomplished by washing the membranes in 500 mL of boiling solution of 0.5% SDS for 1 hour with vigorous shaking. The stripped membranes are stored moist at 4° C. until use.

### References

1. Pugsley M K, Tabrizchi R. The vascular system. An overview of structure and function. J Pharmacol Toxicol Methods. 2000 September-October; 44 (2):333-40.

2. Vane J R, Anggard E E, Botting R M. Regulatory functions of the vascular endothelium. N Engl J Med. 1990 Jul. 5; 323 (1):27-36.

3. Sato Y. Current understanding of the biology of vascular endothelium Cell Struct Funct. 2001 February; 26 (1):9-10.

4. Rivard A, Andres V. Vascular smooth muscle cell proliferation in the pathogenesis of atherosclerotic cardiovascular diseases. Histol Histopathol. 2000 April; 15 (2):557-71.

5. Davies P F. Vascular cell interactions with special reference to the pathogenesis of atherosclerosis. Lab Invest. 1986 July; 55 (1):5-24.

6. Gallicchio M, Argyriou S, Ianches G, Filonzi E L, Zoellner H, Hamilton J A, McGrath K, Wojta J. Stimulation of PAI-1 expression in endothelial cells by cultured vascular smooth muscle cells. Arterioscler Thromb. 1994 May; 14 (5):815-23.

7. Kader K N, Akella R, Ziats N P, Lakey L A, Harasaki H, Ranieri J P, Bellamkonda R V. eNOS-overexpressing endothelial cells inhibit platelet aggregation and smooth muscle cell proliferation in vitro. Tissue Eng. 2000 June; 6 (3):241-51.

8. Xu C B, Stavenow L, Pessah-Rasmussen H. Interactions between cultured bovine arterial endothelial and smooth muscle cells; further studies on the effects of injury and modification of the consequences of injury. Artery. 1993; 20 (3):163-79.

9. Powell R J, Cronenwett J L, Fillinger N W, Wagner R J, Sampson L N. Endothelial cell modulation of smooth muscle cell morphology and organizational growth pattern. Ann Vasc Surg. 1996 January; 10 (1):4-10.

10. Stoll L L, Spector A A. Lipid transfer between endothelial and smooth muscle cells in coculture. J Cell Physiol. 1987 October; 133 (1):103-10.

11. Hay E D. Biogenesis and organization of extracellular matrix. FASEB J. 1999 December; 13 Suppl 2:S281-3.

12. Zamir B, Geiger B. Molecular complexity and dynamics of cell-matrix adhesions. J Cell Sci. 2001 October; 114 (Pt 20):3583-90.

13. Ruoslahti B, Engvall E. Integrins and vascular extracellular matrix assembly. J Clin Invest. 1997 Mar. 15; 99 (6):1149-52.

14. Dogic D, Eckes B, Aumailley M. Extracellular matrix, integrins and focal adhesions. Curr Top Pathol. 1999; 93:75-85.

15. Pauli B U, Knudson W. Tumor invasion: a consequence of destructive and compositional matrix alterations. Hum Pathol. 1988 June; 19 (6):628-39.

16. Batchelor W B, Robinson R, Strauss B H. The extracellular matrix in balloon arterial injury: a novel target for restenosis prevention. Prog Cardiovasc Dis. 1998 July-August; 41 (1):35-49.

17. Wight T N. The extracellular matrix and atherosclerosis. Curr Opin Lipidol. 1995 October; 6 (5):326-34.

18. O'Reilly M S, Boehm T, Shing Y, Fukai N, Vasios G, Lane W S, Flynn E, Birkhead J R, Olsen B R, Folkman J. Endostatin: an endogenous inhibitor of angiogenesis and tumor growth. Cell. 1997 Jan. 24; 88 (2):277-85.

19. Meager A. Cytokine regulation of cellular adhesion molecule expression in inflammation. Cytokine Growth Factor Rev. 1999 March; 10 (1):27-39.

20. Gearing A J, Hemingway I, Pigott R, Hughes J, Rees A J, Cashman S J. Soluble forms of vascular adhesion molecules, E-selectin, ICAM-1, and VCAM-1: pathological significance. Ann N Y Acad Sci. 1992 Dec. 4; 667:324-31.

21. Ross R. Atherosclerosis—an inflammatory disease. N Engl J Med. 1999 Jan. 14; 340 (2):115-26.

22. Ross R. The pathogenesis of atherosclerosis: a perspective for the 1990s. Nature. 1993; 362:801-809.

23. Goldstein J L, Kita T, Brown M S. Defective lipoprotein receptors and atherosclerosis. Lessons from an animal counterpart of familial hypercholesterolemia. N Engl J Med. 1983 Aug. 4; 309 (5):288-96.

24. Haffner S M. Coronary heart disease in patients with diabetes. N Engl J Med. 2000 Apr. 6; 342 (14):1040-2.

25. De Vriese A S, Verbeuren T J, Van de Voorde J, Lameire N H, Vanhoutte P M. Endothelial dysfunction in diabetes. Br J Pharmacol. 2000 130:963-74.

26. Guerci B, Kearney-Schwartz A, Bohme P, Zannad P, Drouin P. Endothelial dysfunction and type 2 diabetes. Part 1: physiology and methods for exploring the endothelial function. Diabetes Metab. 2001 September; 27 (4 Pt 1):425-34.

27. Brownlee M. Biochemistry and molecular cell biology of diabetic complications. Nature. 2001 Dec. 13; 414 (6865):813-20.

28. Brownlee, M., Cerami, A & Vlassara, H. Advanced glycosylation end products in tissue and the biochemical basis of diabetic complications. N. Engl. J. Med. 318, 1315-1320 (1988).

29. Schmidt A M, Yan S D, Wautier J L, Stern D. Activation of receptor for advanced glycation end products: a mechanism for chronic vascular dysfunction in diabetic vasculopathy and atherosclerosis. Circ Res. 1999 84:489-97.

30. Ginsberg H N. Diabetic dyslipidemia: basic mechanisms underlying the common hypertriglyceridemia and low HDL cholesterol levels. Diabetes, 1996 July; 45 Suppl 3:S27-30.

31. Witztum J L, Berliner J A. Oxidized phospholipids and isoprostanes in atherosclerosis. Curr Opin Lipidol. 1998 October; 9 (5):441-8.

32. Festa A, D'Agostino R Jr, Howard G, Mykkanen L, Tracy R P, Haffner S M. chronic subclinical inflammation as part of the insulin resistance syndrome: the Insulin Resistance Atherosclerosis Study (IRAS). Circulation. 2000 Jul. 4; 102 (1):42-7.

1. A method for identifying cell matrix signaling (CMS) pathway induced genes that are modulated during vascular or proliferative diseases and related disorders comprising:
(a) adding one or more vascular disease stimuli to a first cell culture of endothelial cells;
(b) adding one or more vascular disease stimuli to a second cell culture of smooth muscle cells;
(c) adding one or more vascular disease stimuli to a third cell culture of endothelial cells and smooth muscle cells in co-culture;
(d) measuring the amount of vascular disease markers in (a), (b), and (c); and
(e) comparing the amount of the vascular disease markers in (a), (b), and (c) to each other and to controls of untreated cell cultures or co-cultures.

2. The method of claim 1, wherein the vascular disease stimuli are AGE, insulin, IL-1β, or TNF-α or any combination thereof.

3. The method of claim 1, wherein the vascular disease marker is interleukin 6 (IL-6), interferon-inducible protein-10 (IP-10), monokine induced by gamma-interferon (MIG), interferon-inducible T-cell alpha chemoattractant (I-TAC), vascular adhesion molecule-1 (VCAM-1), or monocyte chemoattractant protein-1 (MCP-1).

4. The method of claim 1, wherein the smooth muscle cells are layered over the endothelial cells in co-culture.

5. The method of claim 4, wherein the smooth muscle cells and the endothelial cells are layered in a specific ratio.

6. The method of claim 5, wherein the ratio is 1:1, 1:2, 1:3, or 1:4.

7. A method for identifying compounds that regulate CMS pathway induced genes comprising:
(a) adding one or more vascular disease stimuli and a test compound to a first cell culture of endothelial cells;
(b) adding one or more vascular disease stimuli and the test compound to a second cell culture of smooth muscle cells;
(c) adding one or more vascular disease stimuli and the test compound to a third cell culture of endothelial cells and smooth muscle cells in co-culture;
(d) measuring the amount of vascular disease markers in (a), (b), and (c); and
(e) comparing the amount of the vascular disease markers in (a), (b), and (c) to each other and to endothelial cells, smooth muscle cells, and co-cultures thereof which are untreated, treated with only test compound, or treated with only vascular and proliferative disease stimuli.

8. The method of claim 7, wherein the vascular disease stimuli are AGE, insulin, IL-1β, , or TNFα or any combination thereof.

9. The method of claim 7, wherein the vascular disease marker is IL-6, IP-10, MIG, I-TAC, VCAM-1, or MCP-1.

10. The method of claim 7, wherein the test compound is an oligonucleotide, ribozyme, antisense oligonucleotide, peptide, peptoid, small organic molecule, or small inorganic molecule.

11. The method of claim 10, wherein the test compound is an oligonucleotide complementary to the 5′ region of the CMS pathway induced gene.

12. The method of claim 10, wherein the test compound is a ribozyme molecule that blocks translation of the CMS pathway induced gene.

13. The method of claim 10, wherein the test compound binds the protein product of the CMS induced gene.

14. A method of diagnosing a vascular or proliferative disease or related disorder comprising:
(a) measuring the level of transcription of CMS pathway induced genes present in a patient or patient sample and in a corresponding control sample; and
(b) comparing the level of CMS pathway induced genes transcript in both samples;
wherein if the level of transcript detected differs in the patient sample relative to the corresponding control sample, a vascular disease or a related disorder is diagnosed.

15. A method of diagnosing a vascular or proliferative disease or related disorder comprising:
(a) measuring CMS pathway induced gene product protein level or protein activity present in a patient or patient sample and in a corresponding control sample; and
(b) comparing the CMS pathway induced gene product protein level or protein activity in both samples;
wherein if the level of protein or protein activity detected differs in the patient or patient sample relative to the corresponding control sample, a proliferative disease or an oncogenic related disorder is diagnosed.

16. A method for monitoring the efficacy of treatment for a vascular or proliferative disease or related disorder comprising:
(a) measuring the amount of vascular disease markers in a patient sample from a patient with a vascular disease, proliferative disease, or related disorder;
(b) treating the patient for the vascular or proliferative disease;
(c) measuring the amount of vascular disease markers in patient samples taken during treatment; and
(d) comparing the amount of vascular disease markers in (a) to (c).

17. The method of claim 16, wherein the vascular disease marker is IL-6, IP- 10, MIG, I-TAC, VCAM- 1, or MCP- 1.

18. A method of treating or preventing inflammation comprising administering an effective amount of a pharmaceutical composition comprising one or more compounds determined by the method of claim 7.

19. The method of claim 18, wherein the composition further comprises an auxiliary which is a diluent, binder, stabilizer, buffer, salt, lipophilic solvent, preservative, adjuvant, anti-oxidant, bacteriostat, solute, suspending agent, or thickening agent.

20. The method of claim 18, wherein the composition is in a form suitable for administration by oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transmermal means.

21. The method of claim 18, wherein the composition is in the form of a tablet, capsule, microcapsules, solution, suspension, emulsion, lozenge, cream, gel, paste, pastille, foam, spray, pessary, tamport, or suppository.

22. The method of claim 18, wherein the pharmaceutical composition is administered in combination with other therapeutic agents.

23. A method for regulating protein expression of a CMS pathway induced gene comprising:
(a) identifying one or more compounds which regulate CMS pathway induced genes by the method of claim 7; and
(b) administering an effective amount of a composition containing one or more of said compounds to a patient.

24. A method for identifying compounds that regulate CMS pathway induced gene protein product activity comprising:
(a) adding one or more vascular or proliferative disease stimuli and a test compound to a first cell culture of endothelial cells;
(b) adding one or more vascular or proliferative disease stimuli and the test compound to a second cell culture of smooth muscle cells;
(c) adding one or more vascular or proliferative disease stimuli and the test compound to a co-culture of endothelial cells and smooth muscle cells;
(d) measuring the amount of CMS pathway induced gene protein product activity in (a), (b), and (c); and
(e) comparing the amount of CMS pathway induced gene protein product activity in (a), (b), and (c) to each other and to endothelial cell cultures, smooth muscle cell cultures and co-cultures of endothelial cells and smooth muscle cells which are untreated, treated with only test compound, or treated with only vascular and proliferative disease stimuli.

25. A method for regulating an activity of a protein product of a CMS pathway induced gene comprising:
(a) identifying one or more compounds which affect the activity of CMS pathway induced gene protein products by the method of claim 24; and
(b) administering an effective amount of one or more of said compounds to a patient.

26. A method of diagnosing a vascular or proliferative disease or related disorder comprising:
(a) measuring the level of transcription of CMS pathway induced genes present in a patient sample and in a corresponding control sample; and
(b) comparing the level of CMS pathway induced genes transcript in both samples;
wherein if the level of transcript detected differs in the patient sample relative to the corresponding control sample, a vascular disease or a related disorder is diagnosed.

27. The method of claim 7, wherein the test compound has an unknown effect on smooth muscle cells, an unknown effect on endothelial cells, or an unknown effect on both smooth muscle cells and endothelial cells.

28. The method of claim 1, wherein the one or more vascular disease stimuli is a single vascular disease stimulus.

29. The method of claim 7, wherein the one or more vascular disease stimuli is a single vascular disease stimulus.

30. The method of claim 24, wherein the one or more vascular disease stimuli is a single vascular disease stimulus.
